(19) United States
(12) Patent Application Publication
(10) Pub. No.: US 2016/0163348 A1
(43) Pub. Date: Jun. 9, 2016

(54) DRIVE DEVICE FOR THE BIO-DISC DETECTION

(71) Applicant: QUANTA STORAGE INC., Taoyuan City (TW)

(72) Inventors: Yu-Sheng WANG, Taoyuan City (TW); Chen-Fu CHANG, Taoyuan City (TW); Hsien-Chung OU, Taoyuan City (TW)

(73) Assignee: QUANTA STORAGE INC., Taoyuan City (TW)

(21) Appl. No.: 14/662,921

(22) Filed: Mar. 19, 2015

(30) Foreign Application Priority Data
Dec. 4, 2014 (CN) 201410730118.X

(51) Int. Cl. G11B 19/02; G01N 33/50; G11B 17/028

(52) U.S. Cl. CPC G11B 19/02; G11B 17/0284; G01N 33/50

(57) ABSTRACT

A driver device and a method for bio-disc detection are provided. A spindle motor rotates a bio-disc by a central hole at a high speed. A step motor rotates a periphery of a clamper to rotate the bio-disc at a low speed. When the spindle motor and the step motor work together in conjunction with the separation, mixing and detection process, various rotation modes such as high speed mode, braking mode, direction switching mode and low speed rotation mode can be provided to increase the detection efficiency of the bio-disc.

This application claims the benefit of People's Republic of China application Serial No. 201410730118.X, filed Dec. 4, 2014, the subject matter of which is incorporated herein by reference.

### BACKGROUND OF THE INVENTION

1. Field of the Invention

The invention relates in general to a bio-disc detection system, and more particularly to a driver device used in a bio-disc detection system for rotating a bio-disc to facilitate a detection of a biological sample loaded on the bio-disc.

2. Description of the Related Art

Along with the advance in medical technology, more and more high-speed and precise bio-detection technologies are provided. Bio-disc detection technology, which employs optical detection of biological particles, is capable of concurrently detecting a plurality of biological samples loaded on a disc to automatically and quickly analyzes the samples and has become a main bio-detection technology.

Referring to FIG. 1, a schematic diagram of a bio-disc detection system 10 of the prior art, US Patent Application No. US20030077627, is shown. The bio-disc detection system 10 uses a controller 11 to control a spindle motor 12 to rotate a disc 13. The disc 13 has a plurality of test channels 14 disposed thereon for loading a to-be-tested biological sample and a reagent. The to-be-tested biological sample is such as blood, and the reagent has specific mark such as fluorescence or magnetic beads. Moreover, an optical pick-up head 15 is disposed corresponding to a detection groove of a test channel 14. The optical pick-up head 15 is controlled by a computer 16 to project a light beam, which irradiates the detection groove of the test channel 14. Then, the optical pick-up head 15 and the astigmatism receiver 17 receive a light beam reflected from the detection groove to form an optical signal, which is further transmitted to the computer 16 for detection analysis.

When the bio-disc detection system 10 performs detection, firstly, a to-be-tested biological sample, such as blood, and a reagent are loaded onto respective test channels 14. Then, the controller 11 controls the spindle motor 12 to rotate at a high speed. The disc 13 is rotated at a high speed to generate a centrifugal force enabling cellular pellets to be separated from plasma, wherein cellular pellets and plasma are two ingredients of blood and have different weights. Then, the separated plasma automatically flows to the detection groove to mix up with the reagent. After particles of the plasma, such as pathogens, are marked, the pathogen particles will carry fluorescence or magnetic marks. Then, the number of marked particles in the detection groove can be used as a basis for determining the result of detection. Therefore, during the pre-determined time when the spindle motor 12 rotates, after the plasma and the reagent are fully mixed up, the computer 16 again controls the optical pick-up head 15 to project a light beam to irradiate the detection groove of the test channel 14. Then, the flux of the light is detected to form signals with different intensities for performing detection analysis.

However, the spindle motor 12 of the bio-disc detection system 10 of the prior art is an ordinary driving motor. Although the spindle motor 12 can rotate at a high speed, due to the inertial of rotation, even when the rotation direction of the spindle motor is changed, the bio-disc cannot stop its rotation immediately. It takes a while for the bio-disc to come to a complete stop before the bio-disc can be rotated in a new direction of rotation. Since the bio-disc can neither swing reciprocally to completely mix the plasma with the reagent nor effectively mark the pathogens, the mixing time is increased and the detection efficiency deteriorates. Besides, since the spindle motor of the prior art cannot control the bio-disc to rotate to a predetermined angle at a low speed, the detection groove cannot be precisely positioned, the optical signal cannot be detected, and the reliability of detection result is decreased. Therefore, the bio-disc detection system still has several problems to resolve in the respect of driver device and driving method.

### SUMMARY OF THE INVENTION

According to one embodiment of the present invention, a driver device for bio-disc detection is provided. A step motor rotates a clamper of a bio-disc at a low speed to precisely control a rotation angle of the bio-disc, such that a detection groove on the bio-disc can be correctly positioned and the accuracy of detection can thus be increased.

According to another embodiment of the present invention, a driver device for bio-disc detection is provided. By moving a step motor, a clamper is engaged with or detached from a bio-disc, such that the replacement of the bio-disc is made easier, and the convenience of use is increased.

According to an alternate embodiment of the present invention, a driving method for bio-disc detection is increased. A spindle motor and a step motor work together in conjunction with the separation, mixing and detection process, and various rotation modes can be provided to increase the detection efficiency of the bio-disc.

To achieve the above embodiment of the present invention, a driver device for bio-disc detection is provided. A disc-shaped clamper is disposed on a main body. A cassette mechanism is a frame body disposed inside the main body. A spindle motor is disposed on the cassette mechanism and works together with a clamper to clamp or release a central hole of a bio-disc from atop and underneath. The spindle motor further drives the bio-disc and the clamper by the central hole to rotate at a high speed. The bio-disc has a plurality of test channels and detection groove disposed thereon. A base is disposed on the main body and positioned at the periphery of the clamper. A step motor, rotatably fixed on the base, rotates a driving wheel which is engaged with the periphery of the clamper. A return spring is fixed on the base, wherein one end of the return spring connects and presses the step motor to move the driving wheel towards the periphery of the clamper to rotate the bio-disc.

Each test channel on the bio-disc used in the driver device for bio-disc detection of the present invention has a reagent channel and a sample channel for loading a reagent and a to-be-tested biological sample. The step motor is rotatably fixed on a chute of the base, such that the driving wheel contacts and becomes engaged with the clamper via a belt wheel. Or, the step motor is moved to resist an elastic force of the return spring and drive the driving wheel along the chute to be detached from the periphery of the clamper to release the clamper. Besides, the spindle motor rotates the bio-disc at a high speed to generate a centrifugal force, and the step motor rotates the bio-disc to a predetermined angle at a low speed.

The driving method for bio-disc detection of the present invention comprises following steps. Firstly, a biological sample and a reagent are loaded on a bio-disc, and the detection of the bio-disc starts. Next, a spindle motor is activated, and a separation process is performed in a high-speed rotation mode to separate the biological sample. Then, the spindle motor is turned off, and a step motor is activated to perform a braking process in a braking rotation mode to stop the rotation of the spindle motor. Then, the step motor performs a mixing process in a rotation direction switching mode to mix the biological sample with the reagent. Then, the step motor, in a low-speed rotation mode, drives the bio-disc to pass through the irradiated pre-determined position and performs a detection process to detect an optical signal. Then, the intensity of the detected optical signal is analyzed and a detection result is determined.

The driving method for bio-disc detection of the present invention comprises several rotation modes. In the high-speed rotation mode, the bio-disc is rotated at a high speed to generate a centrifugal force. In the braking rotation mode, the step motor is rotated in a direction inverse to the rotation direction of the spindle motor to brake the spindle motor. Besides, in the rotation direction switching mode, a mixing process is performed to quickly switch the rotation of the step motor between a forward direction and a backward direction, such that the bio-disc wobbles severely and the biological sample and the reagent can thus be mixed completely. In the low-speed rotation mode, the bio-disc is rotated to a predetermined angle, so that the biological sample and the reagent pass through an irradiated pre-determined position at a pre-determined speed or shortly stay at the irradiated pre-determined position to detect an optical signal.

The above and other aspects of the invention will become better understood with regard to the following detailed description of the preferred but non-limiting embodiment(s). The following description is made with reference to the accompanying drawings.

### BRIEF DESCRIPTION OF THE DRAWINGS

FIG. 1 is a schematic diagram of a bio-disc detection system of the prior art;

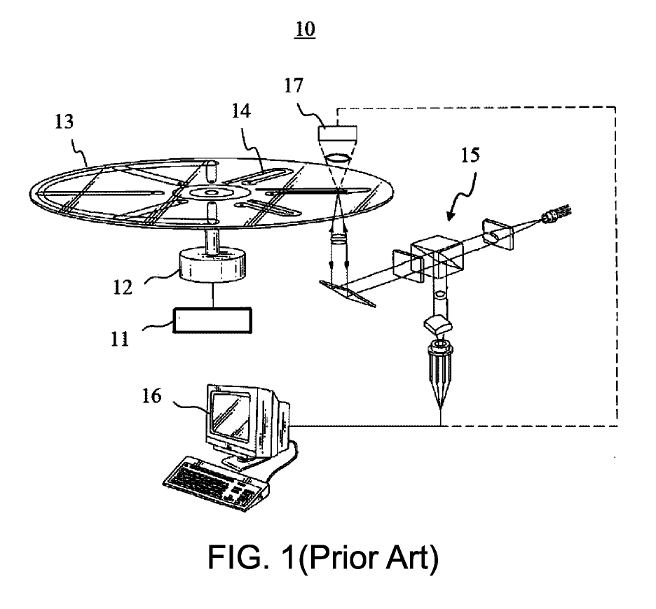

FIG. 2 is a top view of the driver device for bio-disc detection of the present invention in an engaged state;

FIG. 3 is a front view of the driver device for bio-disc detection of the present invention in an engaged state;

FIG. 4 is a front view of the driver device for bio-disc detection of the present invention in a detached state;

FIG. 5 is a functional block diagram of the bio-disc detection system of the present invention;

FIG. 6 is a schematic diagram of rotation mode of the driver device of the present invention; and

FIG. 7 is flowchart of the driving method for bio-disc detection of the present invention.

### DETAILED DESCRIPTION OF THE INVENTION

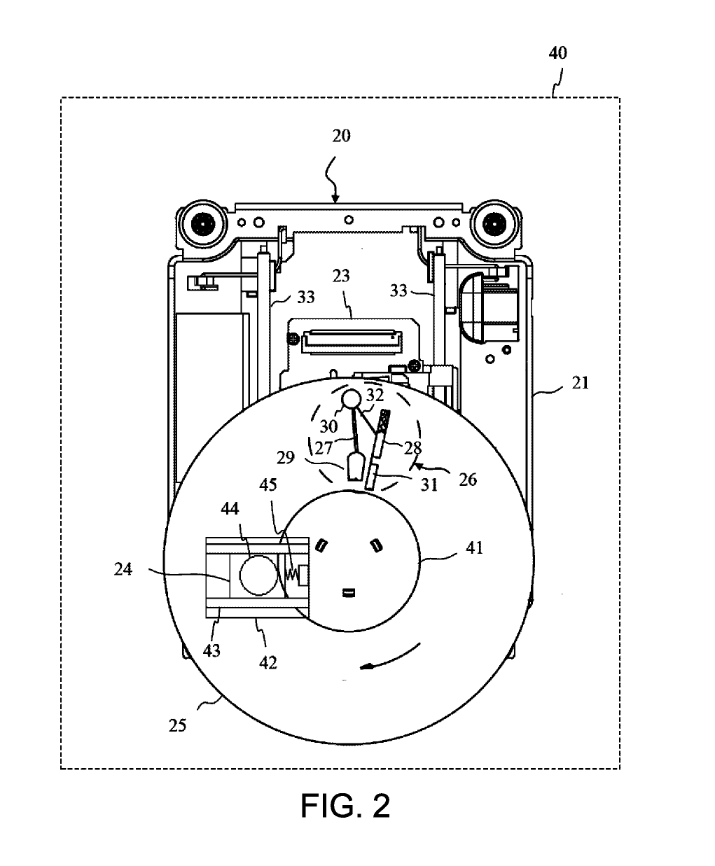

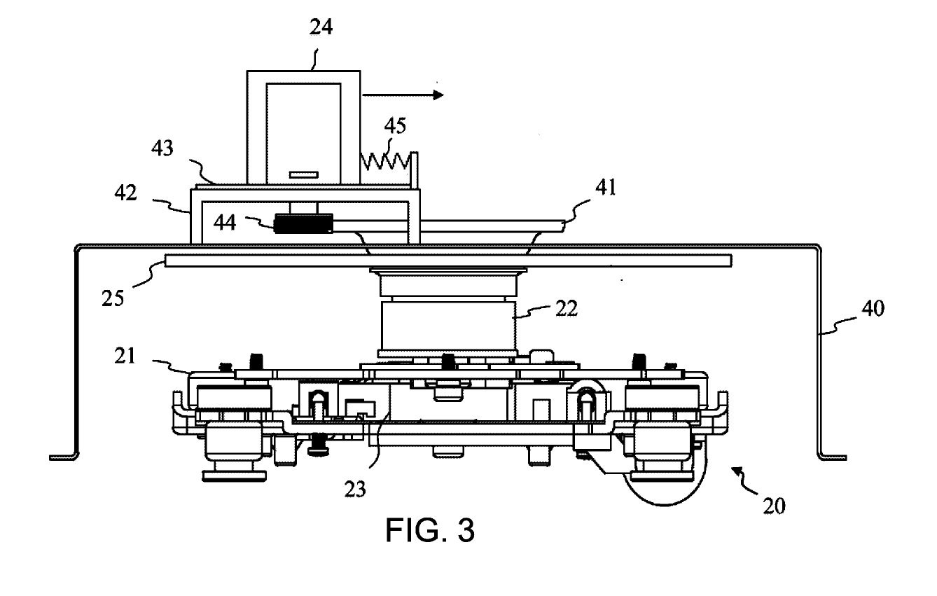

Refer to FIGS. 2 and FIG. 3. FIG. 2 is a top view of the driver device for bio-disc detection of the present invention in an engaged state. FIG. 3 is a front view of the driver device 20 for bio-disc detection of the present invention in an engaged state. The driver device 20 of the present invention is disposed in a main body 40 and mainly comprises a cassette mechanism 21, a spindle motor 22, and a step motor 24. The spindle motor 22 is disposed on the cassette mechanism 21, which is a frame body. The central hole of the bio-disc 25 is mounted on a shaft of the spindle motor 22. The clamper 41 disposed on the main body 40 works with the spindle motor 22 to clamp or release the central hole of the bio-disc 25 as the cassette mechanism 21 ascends or descends. The spindle motor 22 provides a rotational kinetic energy for rotating the clamped bio-disc 25 at a high speed. Meanwhile, the disc-shaped clamper 41 rotates along with the bio-disc 25. The bio-disc 25 has a plurality of test channels 26 disposed thereon. Each test channel 26 has a sample channel 28 and a reagent channel 27 for loading a to-be-tested biological sample and a reagent, respectively. An inlet 29 of the reagent channel 27 is disposed on an inner-ring side of the bio-disc 25, and a terminal end of the reagent channel 27 has a detection groove 30 disposed on an outer-ring side of the bio-disc 25. An inlet 31 of the sample channel 28 is disposed on the inner-ring side of the bio-disc 25, and a terminal end of the sample channel 28 is disposed on the outer-ring side of the bio-disc 25, and is interconnected with the detection groove 30 via a micro-valve channel 32.

The cassette mechanism 21 has an optical pick-up head 23 disposed corresponding to the detection groove 30 of the test channel 26 of the disc 25 for projecting a light beam to irradiate the detection groove 30. Through the guidance of a pair of guide rods 33 disposed on the cassette mechanism 21 in parallel, the optical pick-up head 23 moves along a radial direction of the disc 25 to adjust the position to be irradiated. The main body 40 further has a base 42 disposed outside the periphery of the clamper 41. The base 42 has a chute 43 disposed thereon, wherein the step motor 24 is movably fixed in the chute 43 for rotating a driving wheel 44. One end of the step motor 24 connected to the return spring 45 receives the elastic force of the return spring 45 fixed on the base 42 for pressing the step motor 24 to move towards the periphery of the clamper 41 along the chute 43, such that the driving wheel 44 is engaged with the periphery of the clamper 41 to rotate the clamper 41. Although the engagement between the driving wheel 44 and the periphery of the clamper 41 of the present embodiment is exemplified by the engagement between the driving wheel 44 of the belt wheel and the clamper 41, the engagement is not limited to the belt wheel and can also be done through gear wheels.

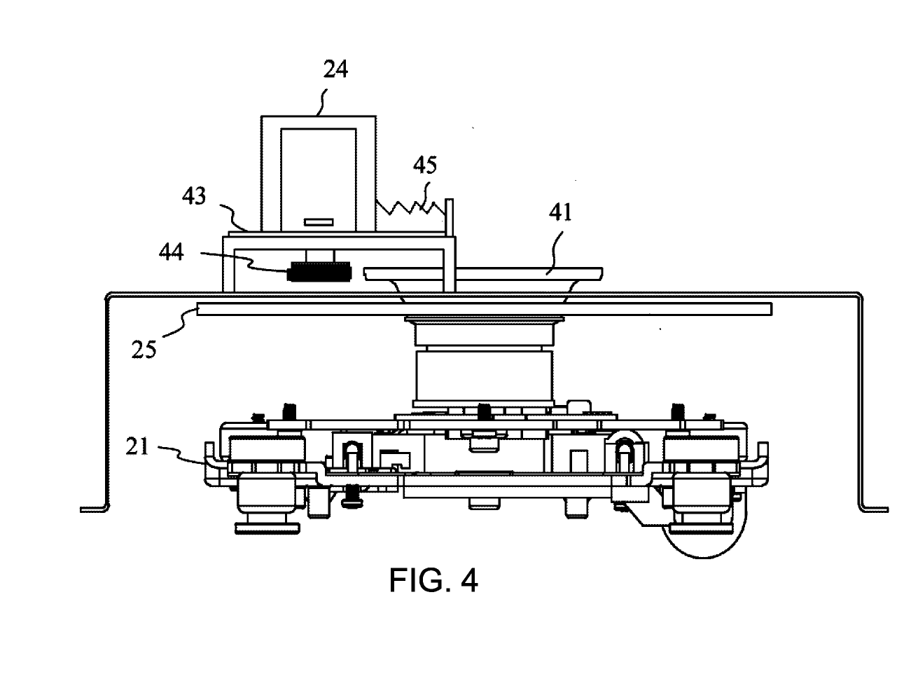

FIG. 4 is a front view of the driver device for bio-disc detection of the present invention in a detached state. When detection is completed or the bio-disc 25 needs to be replaced, the step motor 24 is moved to resist an elastic force of the return spring 45 and drive the driving wheel 44 to move along the chute 43 to be detached from the periphery of the clamper 41 to release the clamper 41. After the clamper 41 is released, the clamper 41 works with the movement of the cassette mechanism 21 to facilitate the replacement of the bio-disc 25. The method of replacing the bio-disc 25 by moving the cassette mechanism 21 is already disclosed in the prior art and does not belong to the technical characteristics of the present invention, and details of the said method are not disclosed here. After the bio-disc 25 is replaced, the step motor 24 is released, such that an elastic force of the return spring 45 of the step motor 24 presses the step motor 24 to move towards the periphery of the clamper 41 along the chute 43 as indicated in FIG. 3 for engaging the driving wheel 44 with the periphery of the clamper 41, and the step motor 24 can rotate the bio-disc 25 via the clamper 41.

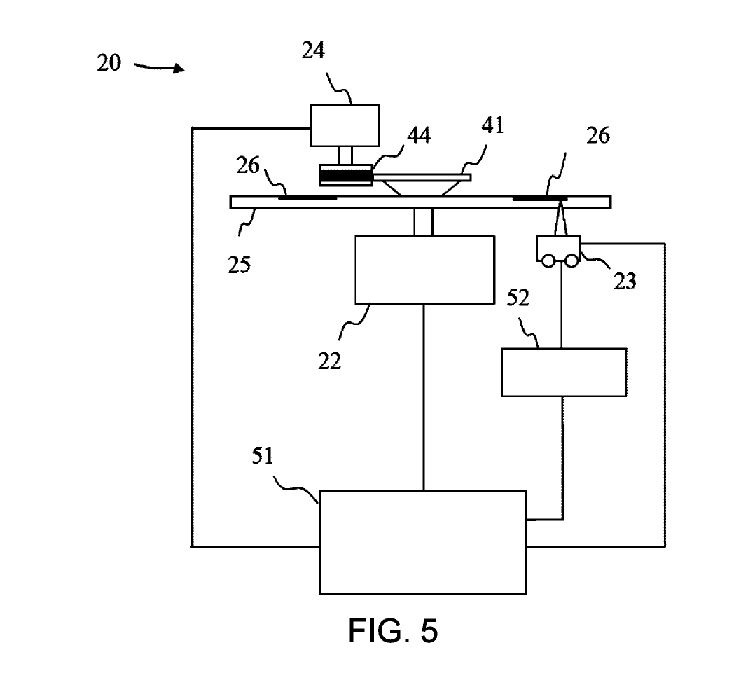

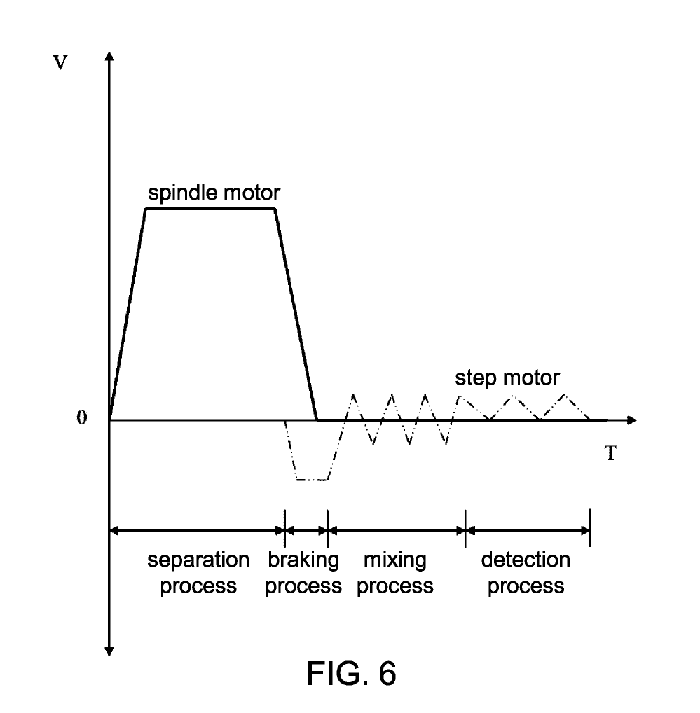

Refer to FIGS. 2 to FIG. 6. FIG. 5 is a schematic diagram of the driver device 20 of the present invention used in a bio-disc detection system 50. FIG. 6 is a schematic diagram of rotation mode of the driver device 20 of the present invention. The bio-disc detection system 50 of FIG. 5 uses a controller 51 to control the rotation of the spindle motor 22 and the step motor 24 of the driver device 20 and the movement of the optical pick-up head 23 and uses the controller 51 to control an analyzer 52 to analyze an optical signal received by the optical pick-up head 23 and determine the result of detection.

When the bio-disc detection system 50 of the present invention detects a biological sample, as indicated in FIG. 4, the step motor 24 is moved to resist the elastic force of the return spring 45 and drive the driving wheel 44 to move along the chute 43 to be detached from the periphery of the clamper 41 to release the clamper 41, such that a new bio-disc 25 can be loaded. As indicated in FIG. 2, after the bio-disc 25 is replaced, the step motor 24 is released, such that the elastic force of the return spring 45 presses the driving wheel 44 to contact the periphery of the clamper 41. In the present embodiment, the biological sample is exemplified by blood which is infused to the inlet 31 of the sample channel 28, and the reagent is infused to the inlet 29 of the reagent channel 27.

Then, the controller 51 activates the spindle motor 22, and in a high-speed rotation mode, after the spindle motor 22 of FIG. 6 is accelerated to a predetermined speed, the bio-disc 25 is rotated to generate a centrifugal force for performing a separation process. In the separation process, the centrifugal force enables the blood and the reagent located on the inner-ring side of the bio-disc 25 to flow to the outer-ring side of the bio-disc 25 along the sample channel 28 and the reagent channel 27 respectively. The reagent flows to the detection groove 30. In the blood, the cellular pellets are heavier than the plasma and are therefore separated from the plasma. The cellular pellets enter the terminal end of the sample channel 28, but the plasma is forced to stay in the middle portion of the sample channel 28. The plasma, being affected by the centrifugal force, enters the detection groove 30 via the micro-valve channel 32 to mix with the reagent which is already in the detection groove 30, such that specific particles of the plasma such as pathogens are marked.

During the separation process, the spindle motor 22 rotates for a pre-determined time in a high-speed rotation mode to fully separate the cellular pellets and the plasma of the blood. Then, the controller 51 turns off the spindle motor 22 and activates the step motor 24 to drive the driving wheel 44 to perform a braking process in a braking rotation mode. During the braking process, after the spindle motor 22a is turned off, the bio-disc 25 still rotates for a while due to the inertia of rotation. Once the step motor 24 is activated, the step motor 24 is rotated in a direction inverse to the rotation direction of the spindle motor 22 and provides a rotational kinetic energy for braking the spindle motor 22. The step motor 24 stops the rotation of the clamper 41 through the driving wheel 44. The clamper 41, formed of a hard material, can directly transmit the rotational kinetic energy of the inverse rotation of the step motor 24 to quickly stop the rotation of the bio-disc 25 and shorten the waiting time required for the bio-disc 25 to stop its rotation.

Then, the step motor 24 performs a mixing process in a rotation direction switching mode by using the characteristics of fast switching of rotation direction. During the mixing process, the step motor 24 of FIG. 6 quickly switches its direction of rotation, the clamper 41, formed of a hard material, transmits the rotational kinetic energy for swinging the bio-disc 25 reciprocally to wobble the detection groove 30 on the bio-disc 25 severely, such that the reagent and the plasma, which are accommodated in the detection groove 30 but are not fully mixed by the centrifugal force in the separation process, can now be fully mixed up and the pathogen particles of the plasma can be completely marked.

After having performed the mixing process for a pre-determined time for fully mixing the plasma with the reagent, the step motor 24 performs a detection process in a low-speed rotation mode. During the detection process, the step motor 24 operates at a lower speed to rotate the clamper 41 through the driving wheel 44 to precisely rotate the bio-disc 25 to a predetermined angle, such that each detection groove 30 passes through a pre-determined position at a predetermined rotation speed or shortly stay at the pre-determined position. Meanwhile, the controller 51 controls the optical pick-up head 23 to project a light beam, which irradiates the detection groove 30, and further moves the optical pick-up head 23 along a radial direction of the guide rod 33 such that the optical pick-up head 23 can irradiate a pre-determined position of the detection groove 30. The optical pick-up head 23 further forms an optical signal according to the intensity of the light reflected from the detection groove 30, and further transmits the optical signal to the analyzer 52. Then, the analyzer 52 analyzes the optical signal and the result of detection is correctly determined.

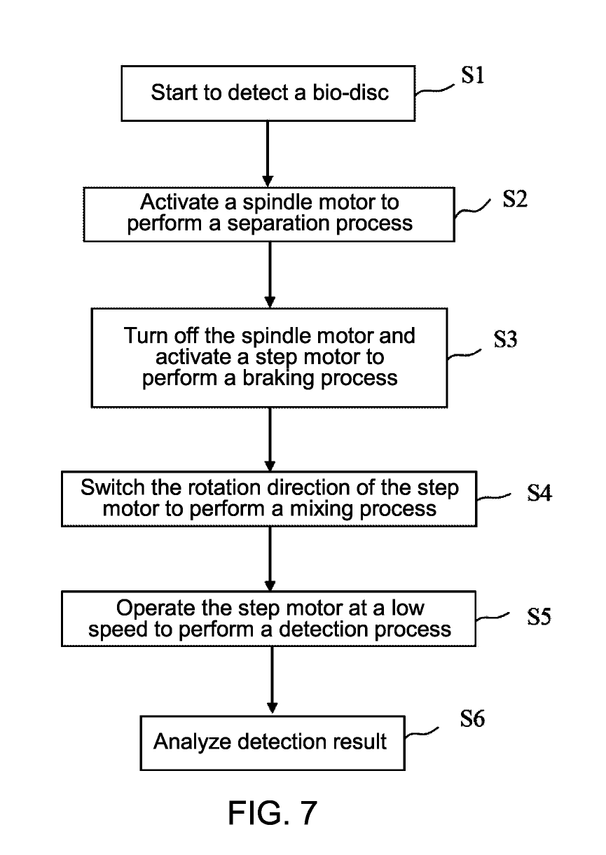

As indicated in FIG. 7, a flowchart of the driving method for bio-disc detection of the present invention is shown. Detailed steps of the driving method for bio-disc detection of present invention are disclosed below. Firstly, the method begins at step S1, a biological sample and a reagent are loaded on a bio-disc, and the detection of the bio-disc starts. Next, the method proceeds to step S2, a spindle motor is activated, and a separation process is performed in a high-speed rotation mode to separate the biological sample. Then, the method proceeds to step S3, after having operated in the high-speed rotation mode for a pre-determined time, the spindle motor is turned off, and a step motor is activated to perform a braking process in a braking rotation mode, and the rotation of the spindle motor is stopped by a rotational kinetic energy generated from the rotation of the step motor. Then, the method proceeds to step S4, after the rotation of the spindle motor is stopped, the step motor performs a mixing process in a rotation direction switching mode to mix the biological sample with the reagent. Then, the method proceeds to step S5, after the mixing process is performed for a pre-determined time, the step motor performs a detection process in a low-speed rotation mode, and precisely rotates the bio-disc to a predetermined angle, such that the biological sample and the reagent, which are mixed up, are driven to pass through a pre-determined position irradiated by the optical pick-up head or shortly stay at the pre-determined position to detect the optical signal. Then, the method proceeds to step S6, the intensity of the detected optical signal is analyzed and a detection result is determined.

According to the driver device for bio-disc detection disclosed in above embodiments of the present invention, through the design enables the step motor to be engaged with or detached from the clamper, the bio-disc can be easily replaced and detected. Furthermore, with the bio-disc being clamped by a clamper, the rotation angle of the bio-disc rotated at a low speed can be precisely controlled, such that the detection groove on the bio-disc can be correctly positioned and the accuracy of detection can be increased. Moreover, the driving method for bio-disc detection of the present invention, the spindle motor and the step motor work together in conjunction with the separation, mixing and detection process, and various rotation modes such as high speed mode, braking mode, direction switching mode and low speed rotation mode can be provided to increase the detection efficiency of the bio-disc.

While the invention has been described by way of example and in terms of the preferred embodiment(s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A driver device for bio-disc detection disposed in a main body, wherein the driver device comprises:
a clamper which is disc-shaped and disposed on the main body;
a cassette mechanism being a frame body;
a spindle motor disposed on the cassette mechanism, wherein the spindle motor works with the clamper to clamp or release a central hole of the bio-disc from atop or underneath, and further drives the bio-disc and the clamper by the central hole to rotate at a high speed, and the bio-disc has a plurality of test channel and detection groove disposed thereon;
a base disposed on the main body and positioned at a periphery of the clamper;
a step motor rotatably fixed on the base, wherein the step motor rotates a driving wheel which is engaged with the periphery of the clamper to rotate the bio-disc; and
a return spring fixed on the base, wherein one end of the return spring connects the step motor and presses the step motor to move the driving wheel towards the periphery of the clamper to rotate the bio-disc.

2. The driver device for bio-disc detection according to claim 1, wherein the test channel has a reagent channel and a sample channel for loading a reagent and a to-be-tested biological sample respectively.

3. The driver device for bio-disc detection according to claim 1, wherein the driving wheel contacts and engages with the clamper via a belt wheel.

4. The driver device for bio-disc detection according to claim 2, wherein the base has a chute disposed thereon and the step motor is rotatably fixed in the chute.

5. The driver device for bio-disc detection according to claim 4, wherein the step motor moves to resist an elastic force of the return spring and drive the driving wheel along the chute to be detached from the periphery of the clamper to release the clamper.

6. The driver device for bio-disc detection according to claim 1, wherein the spindle motor rotates the bio-disc at a high speed to generate a centrifugal force.

7. The driver device for bio-disc detection according to claim 1, wherein the step motor rotates the bio-disc at a low speed.

8. A driving method for bio-disc detection, comprising steps of:
loading a biological sample and a reagent and starting the detection of a bio-disc;
activating a spindle motor and performing a separation process in a high-speed rotation mode to separate the biological sample;
turning off the spindle motor and activating a step motor to perform a braking process in a braking rotation mode to stop the rotation of the spindle motor;
performing a mixing process by the step motor in a rotation direction switching mode to mix the biological sample with the reagent;
driving the bio-disc by the step motor in a low-speed rotation mode to pass through an irradiated pre-determined position and performs a detection process to detect an optical signal;
analyzing an intensity of the detected optical signal and determining a detection result.

9. The driving method for bio-disc detection according to claim 8, wherein in a high-speed rotation mode, the bio-disc is rotated at a high speed to generate a centrifugal force.

10. The driving method for bio-disc detection according to claim 8, wherein after the disc is rotated for a pre-determined time in the high-speed rotation mode, the braking process is performed.

11. The driving method for bio-disc detection according to claim 8, wherein in the braking rotation mode, the rotation of the spindle motor is stopped by a rotational kinetic energy generated from the rotation of the step motor.

12. The driving method for bio-disc detection according to claim 11, wherein in the braking rotation mode, the step motor is rotated in a direction inverse to a rotation direction of the spindle motor to brake the spindle motor.

13. The driving method for bio-disc detection according to claim 8, wherein in the rotation direction switching mode, a rotation of the step motor is quickly switched between a forward direction and a backward direction, such that the bio-disc wobbles.

14. The driving method for bio-disc detection according to claim 8, wherein after the mixing process is performed for a pre-determined time, a detection process is performed.

15. The driving method for bio-disc detection according to claim 8, wherein in the low-speed rotation mode, the bio-disc is rotated by a predetermined angle, so that the biological sample and the reagent, which have been mixed together, pass through the irradiated pre-determined position at a pre-determined speed or shortly stay at the irradiated pre-determined position to detect an optical signal.
